(19) United States
(12) Patent Application Publication
(10) Pub. No.: US 2016/0287192 A1
(43) Pub. Date: Oct. 6, 2016

(54) X-RAY IMAGING DEVICE AND AUXILIARY POSITIONING SYSTEM THEREOF

(71) Applicant: Siemens Healthcare GmbH, Erlangen (DE)

(72) Inventor: Xiao Yun CAI, Shanghai (CN)

(73) Assignee: Siemens Healthcare GmbH, Erlangen (DE)

(21) Appl. No.: 15/034,796

(22) PCT Filed: Oct. 31, 2014
(86) PCT No.: PCT/EP2014/073460

§ 371 Date: May 5, 2016

(30) Foreign Application Priority Data
Nov. 6, 2013 (CN) 201320697395.6

(51) Int. Cl. A61B 6/04; A61B 6/06; A61B 6/00

(52) U.S. Cl. CPC A61B 6/04; A61B 6/464; A61B 6/542; A61B 6/06

(57) ABSTRACT

An X-ray imaging device and an auxiliary positioning system thereof, include a first display, a second display and a camera. The first display is used for displaying a standard position to a patient. The camera is used for acquiring a position image of the patient, and transmitting the acquired position image to the second display. The second display is used for displaying an overlap graph between the position image of the patient and an automatic exposure control domain, as well as a collimator opening domain of a collimator. With the first display displaying a standard position to a patient, the patient may quickly and intuitively know the optimal position, which reduces the time needed for an operator to give explanations to the patient. With the camera acquiring a position image of the patient, and with the second display displaying an overlap graph of the position image of the patient and the like, the operator may conveniently and quickly determine whether the location of the patient is suitable for scanning.

### BACKGROUND OF THE INVENTION

1. Field of the Invention

The present invention concerns a medical apparatus, particularly to an auxiliary positioning system of an X-ray imaging device and the X-ray imaging device.

2. Description of the Prior Art

An X-ray imaging device can be used for irradiating a patient with X-rays so as to obtain the X-ray image of the patient. When using an X-ray imaging device, a patient firstly needs to be positioned, then an operator enters an operation room to take a preview photograph and judge whether the irradiation region is the region required for image formation. If the patient is not accurately positioned, the operator needs to enter the X-ray room to readjust the position of the patient and take a preview photograph again, the above process being repeated until the irradiated region of the patient reaches the optimal position, so as to ensure the optimal image quality. This approach has the following problems. A first is that during the repeated process of taking a preview photograph, a patient cannot avoid being subjected to harm by excessive radiation, which results in an adverse effect on the patient's body. Another problem is that in order to make the irradiated region of a patient reach the optimal position, an operator usually needs to repeatedly go in and out of an X-ray room to adjust the position of the patient, and a patient who lacks professional medical knowledge usually does not know how to cooperate with the operator to better and more quickly reach the optimal position, so that the image formation time is relatively long and the efficiency is relatively low, thereby not only wasting time, but also wasting manpower. A third problem is that if the posing position of a patient cannot properly cover an automatic exposure control domain, the quality of the image obtained by photographing will be influenced.

Chinese patent publication CN 101010039 relates to a control device for an X-ray device and an X-ray device having the control device. The X-ray device has an X-ray generator connected to the control device and at least one camera connected to the control device, wherein the control device is designed to be capable of receiving image data of a patient or an object from the camera. According to the invention, the control device comprises an analysis module, wherein the analysis module is designed to be used for analyzing the image data received from the camera, and generating at least one control signal for controlling the X-ray generator according to one analysis result of the image data, the signal being transmitted to the X-ray generator from the control device.

Chinese patent publication CN 101035466 relates to a method for planning geometric image-formation parameters and an auxiliary system, the geometric image-formation parameters being, for example, a movable dosage measurement domain of an X-ray device (10) and/or an opening of a collimator. The optical image of a patient on a table is generated by a camera and transmitted to a computer. Then the computer overlaps the optical image of the patient and the graphic representation of the geometric image-formation parameters, for example, overlapping with the drawing of an available and/or activated dosage measurement domain. Thus a user can control and interactively select the values of the image formation parameters on an overlap graph.

### SUMMARY OF THE INVENTION

An object of the invention is to provide an auxiliary positioning system of an X-ray imaging device, so as to help a patient rapidly reach the optimal position in the X-ray imaging device, which can reduce the radiation the patient is subjected to and can also increase the efficiency of image formation. A further object is to provide an X-ray imaging device having such an auxiliary positioning system.

In an embodiment of the present invention, an auxiliary positioning system of an X-ray imaging device has a first display, a second display and a camera.

The first display is configured for displaying a standard position to a patient.

The camera is configured for acquiring a position image of the patient, and transmitting the acquired position image to the second display.

The second display is configured for displaying an overlap graph between the position image of the patient and an automatic exposure control domain and/or a collimator opening domain of a collimator.

In another embodiment, the auxiliary positioning system further has an analysis module and a prompt module.

The analysis module is configured for analyzing, according to characteristics of the position image and the collimator opening domain of the collimator, whether a current position of the patient is suitable for exposure, and/or analyzing, according to characteristics of the position image and the automatic exposure control domain, whether a current position of the patient is suitable for exposure, and sending an analysis result to the prompt module.

The prompt module is used for displaying the analysis result of the analysis module to an operator by displaying a prompt message on the second display.

In a further embodiment, if a current position of the patient does not match the collimator opening domain or the automatic exposure control domain, the prompt module is used for prompting the operator by displaying the prompt message on the second display that: the current position of the patient does not match the collimator opening domain or the automatic exposure control domain.

In a further embodiment, the auxiliary positioning system includes an adjustment module.

The analysis module is further configured for obtaining, by analyzing the position image, a scan program initially selected by the operator and the automatic exposure control domain, a body part of the patient which needs to be detected, and analyzing whether the collimator opening domain of the collimator is suitable.

If the size of the collimator opening domain is not suitable for the dimension of the patient, the analysis module notifies the adjustment module of the result; and the adjustment module is used automatically trigger, according to the result, the collimator to adjust the size of its opening so as to fit the dimension of the patient.

The invention also encompasses an X-ray imaging device, including the auxiliary positioning system as described above.

In an embodiment, the X-ray imaging device further has a bracket, an X-ray detector, an X-ray generator, a collimator and a controller.

The X-ray generator is spaced apart from the X-ray detector, and is configured for generating an X-ray beam and irradiating the patient therewith.

The X-ray detector is mounted on the bracket, and is configured for receiving the X-ray penetrating through the patient, and transmitting an image signal to the controller.

The collimator is disposed near the X-ray generator,

The controller is respectively connected to the X-ray detector and the second display of the auxiliary positioning system, and is configured for performing digital processing on the image signal and displaying, on the second display, an image obtained by processing.

The first display of the auxiliary positioning system is disposed on the bracket.

According to an X-ray imaging device and an auxiliary positioning system thereof in an embodiment of the present invention, with a first display displaying a standard position to a patient before starting to scan, the patient may quickly and intuitively know the optimal position, which reduces the time needed for an operator, such as medical care personnel, to give explanations to the patient, thereby not only saving time, but also saving manpower. Additionally a camera is used to acquire a position image of the patient before scanning and transmitting the acquired position image to a second display, thus an overlap graph between the position image of the patient and an automatic exposure control domain, as well as a collimator opening domain of a collimator, is displayed by the second display before scanning, so that the operator may conveniently and quickly determine whether the location of the patient is suitable for scanning, thereby not only reducing the radiation which a patient is subjected to, but also increasing the efficiency of image formation.

The above description is only an overview of the technical solution of the present patent application, in order to more clearly understand the technical means of the present patent application and implement it according to the contents of the description, and in order to make the above and other purposes, characteristics and advantages of the present patent application capable of being more clearly understood, preferred embodiments are given as follows, in combination with accompanying drawings, and the detailed description is as follows.

### BRIEF DESCRIPTION OF THE DRAWINGS

FIG. 1 is a schematic illustration of an X-ray imaging device in one embodiment of the present patent application.

FIG. 2 is a schematic illustration of a first display of an auxiliary positioning system of the X-ray imaging device in FIG. 1, which is displaying a standard position to a patient.

FIG. 3 is an overlap graph between the image of the patient and an automatic exposure control domain, as well as a collimator opening domain of a collimator, which is displayed by a second display of the auxiliary positioning system of the X-ray imaging device in FIG. 1.

### DESCRIPTION OF THE PREFERRED EMBODIMENTS

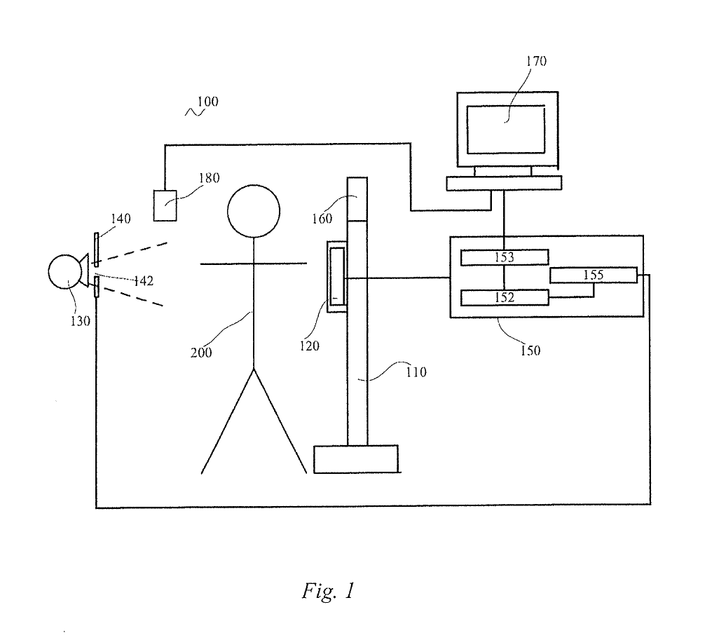

FIG. 1 shows a schematic illustration of an X-ray imaging device 100 in one embodiment of the present patent application. As shown in FIG. 1, the X-ray imaging device 100 includes a bracket 110, an X-ray detector 120, an X-ray generator 130, a collimator 140, a controller 150, a first display 160, a second display 170 and a camera 180, wherein, the first display 160, the second display 170 and the camera 180 are mainly used for the auxiliary positioning function, and therefore may be referred to as an auxiliary positioning system.

The X-ray detector 120 is mounted on the bracket 110, and is for receiving the X-ray penetrating through a patient 200 and transmitting an image signal to the controller 150.

The X-ray generator 130 is spaced apart from the X-ray detector 120, and is for producing X-ray and irradiating the patient 200 with the generated X-ray.

The collimator 140 is disposed near the X-ray generator 130. The collimator 140 has an opening 142. By adjusting the position and size of the opening 142, the irradiated region of the patient can be regulated.

The controller 150 is respectively connected to the X-ray detector 120 and the second display 170, and is used for performing digital processing on the image signal read from the X-ray detector 120 and displaying the photography image of the patient 200 on the second display 170.

The first display 160 is used for displaying a standard position to the patient 200 before working the X-ray generator 130 (i.e. before starting to scan), so that the patient 200 may quickly and intuitively know the optimal position, which reduces the time needed for an operator, such as medical care personnel, to give explanations to the patient 200, thereby not only saving time, but also saving manpower. The first display 160 can be arranged on a position convenient for the patient 200 to observe, such as the bracket 110 and the like.

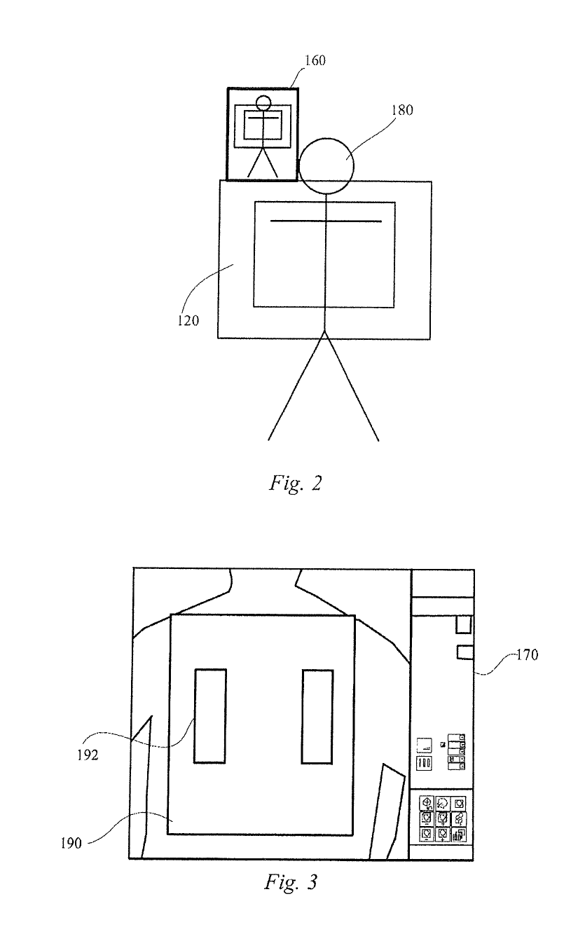

As shown in FIG. 2, the second display 170 is used for displaying an overlap graph between the position image of the patient 200 and an automatic exposure control domain 192, as well as a collimator opening domain 190 of a collimator 140, before scanning. The automatic exposure control domain 192 and the collimator opening domain 190 of the collimator 140 can be obtained from a fuzzy logic control component in the controller 150. According to an overlap graph displayed by the second display 170, an operator can judge whether the location of the patient 200 is suitable for scanning. If not suitable, the operator can make the patient 200 reach an optimal position through a small amount of adjustment, so as to ensure the optimal image quality.

The camera 180 is used for acquiring a position image of the patient 200 before scanning, and transmitting the acquired position image to the second display 170 to display.

In addition, the auxiliary positioning system may further include an analysis module 152 and a prompt module 153. The analysis module 152 and the prompt module 153 may be arranged in the controller 150. The analysis module 152 is used for analyzing, according to characteristics of the position image acquired by the camera 180 and the collimator opening domain 190 of the collimator 140, whether a current position of the patient 200 is suitable for exposure, and sending an analysis result to the prompt module 153. The analysis module 152 is also used for analyzing, according to characteristics of the position image acquired by the camera 180 and the automatic exposure control domain 192, whether a current position of the patient (200) is suitable for exposure, and sending an analysis result to the prompt module 153. The prompt module 153 is used for displaying the analysis result of the analysis module 152 to an operator for the user's reference by displaying a prompt message on the second display 170 and the like before starting to scan. For example, when the position of the patient 200 does not match the collimator opening domain 190 or the automatic exposure control domain 192, the prompt module 153 may prompt a user according to the analysis result received from the analysis module 152: the position is not suitable, whether to continue exposure and the like.

In addition, the auxiliary positioning system may further comprise an adjustment module 155. The adjustment module 155 may be arranged in the controller 150. The analysis module 152 may further obtain, by analyzing the position image acquired by the camera 180, a scan program initially selected by the operator and the automatic exposure control domain 192 and the like, a body part of the patient 200 which needs to be detected, and analyzing whether the collimator opening domain 190 of the collimator 140 is suitable. If the size of the collimator opening domain 190 is not suitable for the dimension of the patient 200, the analysis module 152 may notify the adjustment module 155 of the result, so that the adjustment module 155 can automatically trigger the collimator 140 to adjust the size of the opening 142 so as to fit the dimension of the patient 200.

More specifically, the X-ray imaging device 100 shown in FIG. 1 is illustrated only with a standing-form X-ray imaging device as an example, in other embodiments, the auxiliary positioning system can also be used in a lying-form X-ray imaging device.

As described above, the auxiliary positioning system in one embodiment of the present patent application displays a standard position to the patient 200 through a first display 160 before working the X-ray generator 130, i.e. before starting to scan, so that the patient 200 may quickly and intuitively know the optimal position, which reduces the time needed for an operator, such as medical care personnel, to give explanations to the patient 200, thereby not only saving time, but also saving manpower; meanwhile, the camera 180 is used for acquiring the position image of the patient 200 before scanning and transmitting the acquired position image to the second display 170, thus an overlap graph between the position image of the patient 200 and the automatic exposure control domain 192, as well as the collimator opening domain 190 of the collimator 140, is displayed through the second display 170 before scanning, so that the operator can conveniently and quickly determine whether the location of the patient 200 is suitable for scanning, thereby not only reducing the radiation which the patient is subjected to, but also increasing the efficiency of image formation.

In summary, an X-ray imaging device and an auxiliary positioning system thereof are described herein, including: a first display, a second display and a camera, wherein the first display is used for displaying a standard position to a patient before starting to scan, the camera is used for acquiring a position image of the patient before scanning, and transmitting the acquired position image to the second display, and the second display is used for displaying an overlap graph between the position image of the patient and an automatic exposure control domain, as well as a collimator opening domain of a collimator. With the first display displaying a standard position to a patient before starting to scan, the patient may quickly and intuitively know the optimal position, which reduces the time needed for the operator to give explanations to the patient. With the camera acquiring a position image of the patient and with the second display displaying an overlap graph of the position image of the patient and the like before scanning, the operator may conveniently and quickly determine whether the location of the patient is suitable for scanning.

Although modifications and changes may be suggested by those skilled in the art, it is the intention of the inventor to embody within the patent warranted hereon all changes and modifications as reasonably and properly come within the scope of his contribution to the art.

I claim as my invention:

1. An auxiliary positioning system of an X-ray imaging device, comprising:
a first display configured to display a standard position to a patient;
a second display;
a camera configured to acquire a position image of the patient, and to transmit the acquired position image to the second display; and
the second display being configured to display an overlap graph between the position image of the patient and an automatic exposure control domain and/or a collimator opening domain of a collimator.

2. The auxiliary positioning system as claimed in claim 1, further comprising an analysis module and a prompt module;
wherein the analysis module is configured to analyze at least one of is characteristics of the position image and the collimator opening domain of the collimator to determine whether a current position of the patient is suitable for exposure, and characteristics of the position image and the automatic exposure control domain to determine whether a current position of the patient is suitable for exposure, and to send an analysis result to the prompt module; and
the prompt module is configured to display the analysis result of the analysis module to an operator by displaying a prompt message on the second display.

3. The auxiliary positioning system as claimed in claim 2, wherein the prompt module, if the current position of the patient does not match the collimator opening domain or the automatic exposure control domain is configured to prompt the operator by displaying a prompt message on the second display stating that the current position of the patient does not match the collimator opening domain or the automatic exposure control domain.

4. The auxiliary positioning system as claimed in claim 2, further comprising an adjustment module;
wherein the analysis module is further configured to obtain, by analyzing the position image, a scan program initially selected by the operator and the automatic exposure control domain, a body part of the patient that needs to be detected, and to analyze whether a collimator opening domain of the collimator is suitable; and
if the size of the collimator opening domain is not suitable for the dimension of the patient, the analysis module is configured to notify the adjustment module, and the adjustment module is configured to automatically trigger the collimator to adjust the size of its opening so as to fit the dimension of the patient.

5. (canceled)

6. An X-ray imaging device comprising:
a bracket, an X-ray detector, an X-ray generator, a collimator and a controller;
wherein the X-ray generator is spaced apart from the X-ray detector, and is to generate an X-ray beam that irradiates the patient;
the X-ray detector is mounted on the bracket, and is configured to detect X-rays in said X-ray beam penetrating through the patient, and to transmit an image signal to the controller;
the collimator is disposed near the X-ray generator;
a first display configured to display a standard position to a patient;
a second display;
a camera configured to acquire a position image of the patient, and to transmit the acquired position image to the second display; and
the second display being configured to display an overlap graph between the position image of the patient and an automatic exposure control domain and/or a collimator opening domain of a collimator;
the controller is respectively connected to the X-ray detector and the second display, and is to perform digital processing on the image signal and to display, on the second display, an image obtained by said processing;
and the first display is disposed on the bracket.
